(19) United States
(12) Patent Application Publication
(10) Pub. No.: US 2006/0058672 A1
(43) Pub. Date: Mar. 16, 2006

(54) Expanded performance phased array transducer for a limited number of channels

(76) Inventor: John R. Klepper, Seattle, WA (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 10/917,619

(22) Filed: Aug. 13, 2004

(51) Int. Cl. A61B 8/06

(52) U.S. Cl. 600/447

(57) ABSTRACT

A single transducer, such as a phased array transducer, is provided for various imaging situations with limited loss of resolution and/or penetration. The transducer includes different groups of elements, such as a center group with a fine pitch and outer groups with a larger pitch. The difference in pitch allows for an increased lateral extent of the array, and the fine pitched grouping allows for higher resolution and higher frequency imaging. The same transducer can be used for different imaging situations, such as for neonate imaging as well as imaging 140 pound adults. The difference in pitch also allows for the use of ultrasound imaging systems or beamformers with a limited number of channels even with the total lateral extent provided by the transducer array.

### BACKGROUND

The present invention relates to a phased array transducer for expanded performance despite a limited number of channels. In particular, an increased performance with a lower cost assembly or component transducer is provided for use with an ultrasound system having a limited number of beamformer channels.

Phased array transducers are used for echocardiography with premium ultrasound systems. Relatively long delay lines are provided in order to create beam steering of plus or minus 45° used in echo cardiography. With the advent of low cost digital beamformers within ultrasound systems, phased array imaging may be offered on lower cost platforms.

Lower cost ultrasound imaging systems are typically purchased due to budgetary reasons. The cost of purchasing multiple transducers for different applications may be cost prohibitive. However, physical limitations of phased array imaging may dictate the need for multiple transducers. For example, in pediatric echocardiography, neonates as small as 1 kilogram of weight and with hearts only a few centimeters from a skin surface, but also larger children as large as 50 kilograms at 16 centimeters in depth, may be imaged. Providing high resolution images at depths of a few centimeters for a small neonate and 16 or more centimeters for a larger child requires a broad frequency bandwidth. Using a phased array for color mode imaging may also require broad frequency bandwidth.

Linear and convex transducer arrays may have broader frequency bandwidths. Since linear arrays are used with limited or no steering, broad bandwidth is achieved without concern about acceptance angle or directivity pattern of each element of the transducer. Rather than using the one-half wavelength pitch of phased arrays to avoid grating lobes, a greater wavelength pitch, such as a one or two wavelength pitch, may be used in linear or convex transducer arrays. To further avoid grating lobe noise, the sector scan used for phased arrays is narrowed as the imaging frequency is increased. Since the element pitch is chosen to be one-half the wavelength at the highest frequency to be used, the bandwidth of phased arrays is limited. For use with ultrasound systems having a lower number of channels, such as 64 or 96 channels instead of 128 or 128 channels instead of 192, the lateral extent of an array is limited to the number of channels times one-half the wavelength. This limitation of phased arrays limits both the resolution and penetration at deeper depths even if a low frequency is used because the maximum lateral aperture is small compared to the depth.

### BRIEF SUMMARY

By way of introduction, the preferred embodiments described below include methods, systems and transducers for a broad range of uses in ultrasound imaging. A single transducer, such as a phased array transducer, is provided for various imaging situations with limited loss of resolution and/or penetration. The transducer includes different groups of elements, such as a center group with a fine pitch and outer groups with a larger pitch. The difference in pitch allows for an increased lateral extent of the array, and the fine pitched grouping allows for higher resolution and higher frequency imaging. The same transducer can be used for different imaging situations, such as for neonate imaging as well as imaging 140 pound adults. The difference in pitch also allows for the use of ultrasound imaging systems or beamformers with a limited number of channels even with the total lateral extent provided by the transducer array.

In a first aspect, a system is provided with a transducer for a broad range of uses in ultrasound imaging. An array of elements extends along at least one dimension. The array has two groups of adjacent elements. The first group has a different pitch than the second group. A beamformer is connectable with the elements of the array. The beamformer is operable to connect with elements of both the first and second groups at a same time while steering away from orthogonal to the array.

In a second aspect, a method is provided for ultrasound imaging with a transducer operable for a broad range of uses. Signals corresponding to an acoustic beam are steered away from orthogonal to an array of elements. The steering of the signals is applied to first and second groups of contiguous elements. The first group has a different pitch than the second group.

In a third aspect, a transducer is provided for a broad range of uses in ultrasound imaging. An array of elements has a uniform pitch along a lateral dimension. The array includes two groups of contiguous elements. The contiguous elements of a first group are electrically isolated from each other. At least pairs of the contiguous elements of the second group are substantially permanently electrically connected.

The present invention is defined by the following claims, and nothing in this section should be taken as a limitation on those claims. Further aspects and advantages of the invention are described below in conjunction of the preferred embodiments and may be later claimed independently or in combination.

### BRIEF DESCRIPTION OF THE DRAWINGS

The components and the figures are not necessarily to scale, emphasis instead being placed upon illustrating the principles of the invention. Moreover, in the figures, like reference numerals designate corresponding parts throughout the different views.

FIG. 1 is a block diagram of one embodiment of a system with a transducer for a broad range of uses in ultrasound imaging; and

FIG. 2 is a flow chart diagram of one embodiment of a method for ultrasound imaging with a transducer operable for a broad range of uses.

### DETAILED DESCRIPTION OF THE DRAWINGS AND PRESENTLY PREFERRED EMBODIMENTS

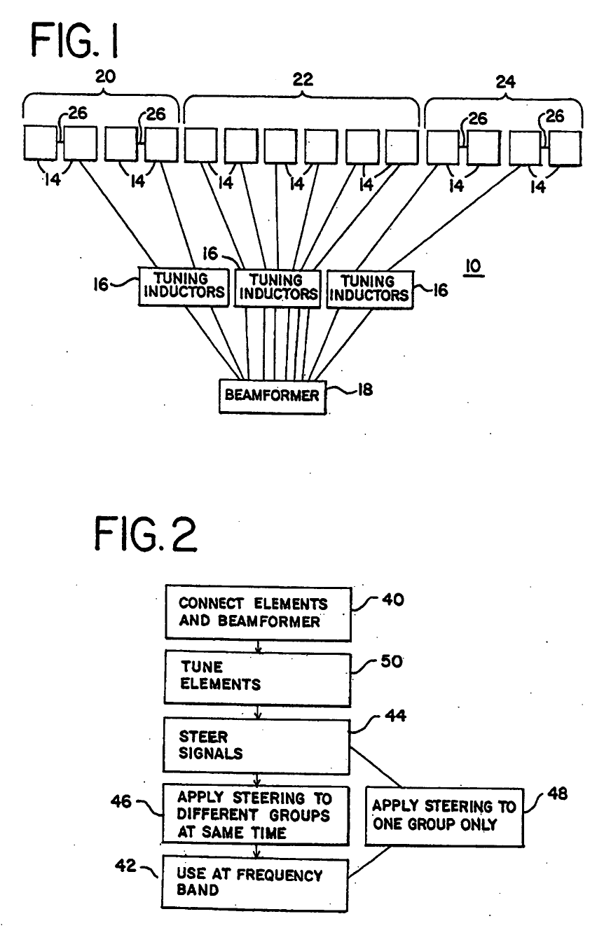

FIG. 1 shows a system 10 with a transducer 12 for ultrasound imaging. The transducer 12 includes an array of elements 14. The system 10 also includes tuning inductors 16 and a beamformer 18. Additional, different or fewer components may be provided. In one embodiment, the system 10 is a low cost ultrasound imaging system, such as a system having 64, 96, or 128 channels. Greater or fewer number of channels may be used. In alternative embodiments, a high cost ultrasound imaging system having any number of channels is used. The transducer 12 permanently or detachably connects with the beamformer 18. The transducer 12 is connected to the beamformer 18 for imaging at both near field (e.g., 1-3 centimeters) or far field (14-16 centimeters) objects. The same transducer is used for imaging prenatal infants as well as full grown adults, but lesser range of sizes may be provided.

The elements 14 are piezoelectric ceramic, piezoelectric composite, capacitive membrane, microelectromechanical, combinations thereof or other now known or later developed devices for transducing between electrical and acoustical energies. Each of the elements 14 is associated with a plurality of electrodes, such as a grounding plane on one side and an electrical connection with system or beamformer channels on another side. Matching layers, backing block and/or lens material may be provided. The elements 14 are positioned within a transducer probe housing, such as a handheld, a catheter, a transesophageal, an endocavity, or any other or now known or later developed probe housings for use internally or externally to a patient. The housing connects through one or more cables, such as coaxial cables, to a connector for connection with an imaging system.

The elements 14 are configured as an array. For example, a one-dimensional array of elements 14 extends along a lateral dimension. 1.25, 1.5, 1.75 and 2-dimensional arrays may be used in other embodiments. The arrangement of the elements 14 in the array provides for different pitches for different groups 20, 22 and 24 of elements 14. While three groups are shown, two or four or more groups of elements 14 associated with different pitches may be used. Some groups 20, 22, 24 may have a same pitch as other groups but different than at least one group 20, 22, 24 of elements 14. Different relative positional arrangements may be used. Each of the groups 20, 22, 24 may have a same or different number of elements 14 as other groups 20, 22 and 24. Any of various different pitches may be used, such as having one group 24 with twice the pitch of another group 22. Other integer or fractional pitch differences may be used.

In one embodiment, the difference in pitch is obtained by having elements 14 of different sizes or with different spacing between the elements 14. For example, the elements 14 of the center group 22 have a fine pitch, and the elements 14 of the outer groups 20 and 24 are rectangular elements with a larger lateral extent or a greater gap between elements 14 than for the elements 14 of the center group 22.

In the embodiment shown in FIG. 1, the elements 14 have a uniform pitch along the lateral dimension. The array of elements 14 is configured as a phased array. The uniform pitch corresponds to one-half a wavelength of a higher or highest ultrasound frequency within a range of possible operation of the transducer. For example, the transducer is operated within a 3.5-9, 4-8, 4-9.5 MHz or other frequency ranges. Any of various bandwidths may be provided, such as a 65%, 70% or larger or smaller bandwidths. For example, a 5.5 megahertz bandwidth is provided for a transducer with a 7 megahertz center frequency for cardiac imaging. The bandwidth at −6 dB down is provided from 3.5 megahertz to 9 megahertz. A uniform pitch of 0.12 millimeters is used for elements 14 across the entire array. The length or extent along the lateral dimension of the elements 14 is a function of the lowest or one of the lower ultrasound frequencies within the range of possible operation of the transducer 12. The total lateral aperture is determined from the lowest frequency of interest in order to achieve the deepest depth penetration and resolution. The pitch is chosen to achieve good high frequency response. Uniform dicing to provide the uniform pitch of the transducer elements 14 may reduce manufacturing costs as compared to non-uniform pitch of the elements 14.

Even with uniformly pitched elements 14, a different pitch is provided within the different groups 20, 22 and 24 of contiguous or adjacent elements 14. While groups 20 and 24 shows four contiguous or adjacent elements 14, a greater or fewer number of elements 14 may be provided. Group 22 includes six elements 14 as shown but may include fewer or greater number of elements 14. While 14 elements 14 are shown, a greater or less number of elements 14 may be provided, such as 28, 32, 64, 96 or 128. To obtain a finer pitch for the center group 22, each of the elements 14 is electrically isolated from each other. For a larger or coarser pitch in the other outer groups 20, 24, at least pairs of the contiguous elements 14 are electrically connected together. As shown in FIG. 1, adjacent pairs of elements 14 are connected together by the connectors 26. Different numbers of elements may be connected together within a group, such as every three elements, every four elements or other number of elements. Non-adjacent elements 14 within a contiguous group 20, 22, 24 may be connected together in yet other embodiments.

The connector 26 is a substantially permanent electrical connection in one embodiment. Substantially permanent is used to account for a non-switched structure, such as a wiring together of elements. Stress or purposeful removal of the permanent connection by disassembly of the transducer 12 may be possible while still being substantially permanent. For example, the connected elements 14 are hardwired together using a wire bond, a flexible circuit connection, an electrical trace deposition between associated electrodes, a common electrode, combinations thereof or other now known or later developed technique for connecting two acoustically separated elements together electrically. In alternative embodiments, the electrical connection 26 is provided through switches, such as a multiplexer. The multiplexer may be positioned within the transducer 12 or within an imaging system or the beamformer 18.

As an alternative to the connector 26, the different pitch of the groups 20, 22, 24 is provided by using full sampling for the group 22 and sparse sampling for the groups 20 and 24. For example, channels of the beamformer 18 connect to every other or every third element 14 within groups 20 and 24 and connect to every element 14 of the group 22. Sparse sampling may be used across the entire array with sparser sampling for one group 20, 22, 24 than another group 20, 22, 24.

Different groups of elements 20, 22 and 24 have different ratios of number of elements 14 to number of output channels, such as coaxial cables, flex circuits or other channels output from the transducer 12 for connection with the beamformer 18. The total number of channels connectable with the beamformer 18 is less than the total number of elements 14 due to the pitch difference. Where the connectors 26 are provided, the channels connect with all of the elements 14, such as connecting directly to a connector which then electrically connects with two different elements 14, connecting to one element 14 and the other element through the connector 26 and/or the channel connecting to two elements 14. For example, an array of 96 elements 14 is provided with a uniform pitch of 0.12 millimeters. The resulting breadth of the array along the lateral dimension is about 11.4 millimeters. For connection with a 64 channel beamformer, the center 32 elements 14 are used to form group 22 for connection with 32 channels. The outer 16 elements 14 of groups 20 and 24 are connected in pairs to provide another 32 electrical channels or elements. The entire breadth of the array is used with a fewer number of channels than elements 14.

The beamformer 18 is a transmit beamformer and/or a receive beamformer. The beamformer 18 includes application specific integrated circuits, processors, filters, delays, summers, phase rotators, amplifiers, waveform generators, memories, analog circuits, digital circuits, combinations thereof or other now known or later developed receive or transmit beamformer component. The beamformer 18 is configured into a plurality of beamformer or system channels. For example, a transmit beamformer has a plurality of channels for the generation of relatively delayed and apodized waveforms. As another example, a receive beamformer has a plurality of channels for applying relative delays and apodization followed by summation across the channels to generate data representing a spatial location along a scan line. The relative delays and apodization focus the acoustic energy along one or more scan lines. The beamformer 18 is connectable directly or indirectly with the elements 14 of the array. The beamformer 18 is operable to connect with elements of all, a plurality or only one of the groups at a same time. For example, channels of the beamformer 18 connect with channels of the transducer 12 across the entire array of elements 14. Using relative delays and apodization, acoustic energy may be steered within a scan region, such as steering the acoustic energy away from orthogonal to the array. The connection is provided at a same time so that steering may be applied during a receive or transmit event. In alternative embodiments, the steering is orthogonal to the array. Any angles or ranges of steering may be used, such as performing a sector scan of plus or minus 45° as the maximum angles.

The number of channels of the beamformer 18 connected with channels of the transducer 12 is based on the number of available channels, the pitch of the elements 14 or groups 20, 22, 24, and the size of the aperture of the transducer 12. For example, the groups 20 and 24 each have four elements 14. The group 22 includes six elements 14. The number of elements in one group may be the same or not equal to the number of elements in another group. The beamformer 18 as shown in FIG. 1 includes 10 channels. Six of the 10 channels connect with the six elements 14 of the center group 22. Two of the channels connect with two pairs of elements 14 of the group 20, and another two channels connect with the pairs of elements 14 of the group 24. The increase in pitch associated with the outer groups 20 and 24 allows for a fewer number of beamformer channels than elements 14 without or with minimal sacrifice of lateral aperture.

The beamformer 18 is operable over a range of ultrasound frequencies. The filters, waveform generators and other components of the beamformer 18 are operable over a desired range of frequencies, such as from 1-12, 3-9, 4-8, 1-6, 5-12 MHz or other range of frequencies and given center frequencies. The beamformer 18 may be operable at one range for transmit and operable at a fractional harmonic, subharmonic or integer harmonic range of frequencies for receive. The range of operation of the beamformer 18 is generally matched to the range of operation of the transducer 12.

In one embodiment, the uniform pitch structure of the transducer 12 corresponds to one-half the wavelength of one of the higher of the ultrasound frequencies within the range of operation of the transducer 12 or beamformer 18. For example, the uniform pitch corresponds to one-half the wavelength of a highest ultrasound frequency to be used. The transmit or receive aperture, such as the entire length of the array of elements 14 along the lateral dimension is a function of one of the lower of the ultrasound frequencies of the range. For example, the lowest ultrasound frequency within the desired range is used to determine the number of elements 14, the pitch and/or the extent of the elements 14 along the lateral dimension.

Using control of the beamformer 18, a multiplexer, switching or other connections of the beamformer 18 to the transducer 12, the beamformer 18 is operable to use different groups 20, 22, 24 and/or different elements 14 for different imaging situations. For example, the beamformer 18 is operable to use the center elements 14 with the fine pitch for shallow field imaging without using the outer groups of 20, 24 of elements 14. Any range of depth may be considered shallow field imaging, such as 1-3 centimeters. All of the groups or two of the groups 20, 22 and 24 are used for far field imaging, such as greater than three centimeters. All of the elements 14 are used for the far field imaging, but fewer than all the elements 14 may be used, such as not using the outermost elements 14. Different focal depths and associated mixture of different elements 14, different groups 20, 22, 24 and/or different aperture sizes may be used. The amount of steering may also dictate the number of elements 14, the groups 20, 22, 24 or combinations thereof used. For example, for steering angles away from orthogonal, such as greater than 20 degrees, all of the elements 14 associated with the course pitch are excluded from the aperture to avoid grading lobes. The groups 20, 24 associated with a courser pitch may be used where needed due to depth and/or a decrease in frequency. A variable frequency filter within the beamformer 18 may also be used to remove grading lobe artifacts. Alternatively, apodization of the outside elements may be used to reduce the contribution of outside elements to the transmit and/or receive beams.

The tuning inductors 16 are series inductors positioned within the transducer 12, a connector between the transducer 12 and the beamformer 18 or within the beamformer 18. In one embodiment, different tuning inductors 16 are provided for different ones of the elements 14. For example, tuning inductors 16 associated with the finer pitch of the group 22 are selected based on the highest or one of the higher ultrasound frequencies to be used. The tuning inductor 16 associated with the courser pitch of the groups 20, 24 are selected as appropriate for a lowest or lower frequency of operation. In an alternative embodiment, all of the tuning inductors 16 are the same for each of the groups 20, 22, 24, such as a 2.2 μH series inductor for each channel.

FIG. 2 shows one embodiment of a method for ultrasound imaging with a transducer operable for a broad range of uses. Additional, different, or fewer acts may be provided in the same or a different order shown in FIG. 2. For example, one of acts 46 or 48 is skipped. The system of FIG. 1 or a different system is used to implement the method of FIG. 2.

In act 40, interconnection between elements 14 and the beamformer is performed. For example, adjacent elements within a group of elements are substantially permanently connected together. During manufacture or by nature of the type of manufacture, adjacent pairs, triplets, quadruplets or other size of uniformly spaced elements are connected together to increase a pitch associated with each beamformer channel. Alternatively, sparse sampling or element size/gap is used. The connection of elements is performed for a portion of the array or differently for different portions of the array. The connected elements as well as elements free of electrical connection are connected with a beamformer. For example, a permanent connection is formed with a beamformer during manufacture. As another example, channels of a beamformer are connected with channels of a transducer by plugging a transducer connector or cable into an imaging system. Set connections are used or a multiplexer establishes a connection of specific beamformer channels to specific transducer element channels.

In act 50, the elements used for transducing between the acoustic and electrical energies or signals are tuned. The same tuning may be used for each of the elements. Alternatively, elements of one group are tuned differently than elements of another group. Tuning is provided by passive components, such as inductors or capacitors. Alternatively, active components may be used for tuning the elements.

In act 44, the signals are steered. Signals are provided at the selected frequency band or frequency bands. For sector or other phased array imaging, the signals on transmit or receive are steered along an acoustic beam away from orthogonal to the array of elements. For example, a sector image is scanned over a 90° steering range. The transmitted or received acoustic beam has an origin on the face of the transducer array. Signals steered away from orthogonal may have a substantial angle to the orthogonal, such as 5°, 10°, 20° or more. Where the array is curved, the orthogonal is determined at the origin of acoustic beam on the transducer array. In addition or alternative to steering the signals away from orthogonal, signals may be steered along the orthogonal in other transmit or receive events for scanning a region.

In act 46, the steering of act 44 is applied to different groups of contiguous elements at a same time or during a same transmit or receive event. One group of elements has a different pitch than the other group of elements. In the system of FIG. 1, the steering is applied to three groups of contiguous elements. A center group has a finer pitch than the outer two groups. With a uniform pitch structure of the elements, the different pitch within the groups of elements relative to other groups of elements corresponds to the connection of adjacent elements together within a group. Alternatively, the steering to elements with different pitches is provided. In one embodiment, any scanning uses all the elements or groups of elements with different pitches. In other embodiments, the application of steering to multiple groups associated with different pitches in act 46 is performed for far field imaging. The extent of inclusion of elements associated with different or greater pitch may depend on the depth of focus or frequency being used.

In act 48, the steering of act 44 is applied to only one group of elements, such as a center group of elements associated with a finer or finest pitch. For example, the center group of elements with the finer pitch is used for high frequency low depth imaging or shallow field imaging.

The electrical signals for transmission of acoustic waveforms or for representing receive echoes are used at a desired frequency brand within a range of possible frequencies in act 42. For example, the lateral extent of the elements of the transducer 12, the pitch of the elements and/or other characteristic of the transducer or beamformer provides for the possible range of frequencies that may be used. A desired frequency is selected as a function of the scan type (i.e. sector versus linear scanning), the depth of focus, the desired resolution, desired center frequency and/or other characteristics. Given a uniform pitch structure of elements, the pitch may determine the highest or a higher ultrasound frequency of the possible range, such as the pitch corresponding to one-half the wavelength of the highest frequency to be used. The lateral extent of the elements of the array may limit the lower or lowest ultrasound frequencies within the possible range. In response to user setting of the focal depth, user selection of an imaging application, transducer identification provided automatically by the transducer or input by the user, or other input, the beamformer parameters including the center frequency and frequency bandwidth for imaging are configured within the beamformer 18.

While the invention has been described above by reference to various embodiments, it should be understood that many changes and modifications can be made without departing from the scope of the invention. It is therefore intended that the foregoing detailed description be regarded as illustrative rather than limiting, and that it be understood that it is the following claims, including all equivalents, that are intended to define the spirit and scope of this invention.

I claim:

1. A system with a transducer for a broad range of uses in ultrasound imaging, the system comprising:
an array of elements extending along at least one dimension, the array having first and second groups of adjacent elements, the first group of elements having a different pitch than the second group of elements; and
a beamformer connectable with the elements of the array, the beamformer operable to connect with elements of both the first and second groups at a same time while steering away from orthogonal to the array.

2. The system of claim 1 wherein the array of elements comprises a phased array.

3. The system of claim 1 wherein the array of elements has a third group of adjacent elements, the first group of elements having a finer pitch than the second and third groups of elements, the first group of elements positioned between the second and third groups of elements along the at least one dimension.

4. The system of claim 1 wherein the elements of the array have a uniform pitch structure wherein the different pitch of the second group of the elements relative to the first group of elements corresponds to connecting together adjacent elements within the second group.

5. The system of claim 4 wherein the adjacent elements within the second group are substantially permanently connected together.

6. The system of claim 4 wherein the first group of elements includes N elements and the second group of elements includes M elements, N being equal or non-equal to M, and wherein the beamformer comprises P channels, N of the P channels connected to respective ones of the N elements of the first group and M/2 of the P channels connected to respective pairs of the connected together adjacent elements within the second group, P being less than a total number of elements.

7. The system of claim 4 wherein the beamformer is operable over a range of ultrasound frequencies, the uniform pitch structure corresponds to a one-half wavelength of one of a higher of the ultrasound frequencies within the range.

8. The system of claim 1 wherein the elements of the array have a uniform pitch structure wherein the different pitch of the second group of the elements relative to the first group of elements corresponds to a full sampling connection of channels of the beamformer to the elements of the first group and a sparse sampling connection of channels of the beamformer to the elements of the second group.

9. The system of claim 1 wherein the beamformer is operable over a range of ultrasound frequencies, the extent of the elements along the at least one dimension being a function of one of a lower of the ultrasound frequencies within the range.

10. The system of claim 1 wherein the beamformer is operable over a range of ultrasound frequencies, the uniform pitch structure corresponds to a one-half wavelength of one of a higher of the ultrasound frequencies within the range, the extent of the elements along the at least one dimension being a function of one of a lower of the ultrasound frequencies within the range;
further comprising:
first tuning inductors connected with respective elements of the first group of elements, an inductance of the first tuning inductors being a function of the higher frequency; and
second tuning inductors connected with respective elements of the second group of elements, an inductance of the second tuning inductors being a function of the lower frequency.

11. The system of claim 1 wherein the beamformer is operable to use the first group of elements alone for shallow field imaging and is operable to use both the first and second groups of elements for a far field imaging.

12. A method for ultrasound imaging with a transducer operable for a broad range of uses, the method comprising:
(a) steering signals corresponding to an acoustic beam away from orthogonal to an array of elements; and
(b) applying the steering of (a) to first and second groups of contiguous elements, the first group of elements having a different pitch than the second group of elements.

13. The method of claim 12 wherein (a) comprises steering 20 degrees or more from orthogonal to the array of elements at the origin of the acoustic beam.

14. The method of claim 12 wherein (b) comprises applying the steering of (a) to the first, second and third groups of contiguous elements, the first group of elements having a finer pitch than the second and third groups of elements, the first group of elements positioned between the second and third groups of elements along a at least one dimension of the array.

15. The method of claim 12 wherein (b) comprises applying the steering where the elements of the array have a uniform pitch structure, the different pitch of the second group of the elements relative to the first group of elements corresponding to connecting together adjacent elements within the second group.

16. The method of claim 15 further comprising:
(c) substantially permanently connecting together adjacent elements of the second group.

17. The method of claim 15 wherein the first group of elements includes N elements and the second group of elements includes M elements, N being equal or non-equal to M, and wherein (b) comprises connecting P channels to the elements of the array, N of the P channels connected to respective ones of the N elements of the first group and M/2 of the P channels connected to respective pairs of the connected together adjacent elements within the second group, P being less than a total number of elements.

18. The method of claim 15 further comprising:
(c) using the signals at one frequency band within a range of possible ultrasound frequencies, the uniform pitch structure corresponding to a one-half wavelength of one of a higher of the ultrasound frequencies within the range.

19. The method of claim 12 further comprising:
(c) using the signals at one frequency band within a range of possible ultrasound frequencies, a lateral extent of the elements of the array being a function of one of a lower of the ultrasound frequencies within the range.

20. The method of claim 12 further comprising:
(c) tuning the elements of the first group differently than the elements of the second group.

21. The method of claim 12 wherein (a) and (b) are performed for far field imaging;
further comprising:
(c) using the first group of elements without the second group of elements for shallow field imaging.

22. A transducer for a broad range of uses in ultrasound imaging, the transducer comprising:
an array of elements with uniform pitch along a lateral dimension, the array having first and second groups of contiguous elements, the contiguous elements of the first group being electrically isolated from each other, at least pairs of the contiguous elements of the second group being substantially permanently electrically connected.

23. The transducer of claim 22 wherein the array of elements comprises a phased array.

24. The transducer of claim 22 wherein the array of elements has a third group of contiguous elements, the first group of elements having a finer pitch than the second and third groups of elements, the first group of elements positioned between the second and third groups of elements along the lateral dimension.

25. The transducer of claim 22 wherein a total number of channels connectable with a beamformer is less then a total number of elements, the channels being connected with all of the elements.

26. The transducer of claim 22 wherein the uniform pitch corresponds to a one-half wavelength of one of a higher of ultrasound frequencies within a range of possible operation of the transducer.

27. The transducer of claim 22 wherein an extent of the elements along the lateral dimension is a function of one of a lower of ultrasound frequencies within a range of possible operation of the transducer.
